(19) United States
(12) Patent Application Publication
(10) Pub. No.: US 2010/0159616 A1
(43) Pub. Date: Jun. 24, 2010

(54) Bio-Chip of Pattern-Arranged in Line, Method for Manufacturing the Same, and Method for Detecting an Analyte Bound in the Same

(75) Inventors: Kwan Han Park, Incheon (KR); Kang Hoon Lee, Gyeonggi-do (KR); Byong Chon Jon, Gyeonggi-do (KR); Moon Hi Han, Seoul (KR); Yoon Suk Lee, Gangwon (KR)

Correspondence Address: NIXON PEABODY, LLP, 401 9TH STREET, NW, SUITE 900, WASHINGTON, DC, 20004-2128, US

(73) Assignee: KOREA TECHNOLOGY INDUSTRY CO., LTD., Seoul (KR)

(21) Appl. No.: 11/883,931

(22) PCT Filed: Apr. 16, 2007
(86) PCT No.: PCT/KR2007/001831

§ 371 Date: Aug. 8, 2007

(30) Foreign Application Priority Data
Apr. 17, 2006 (KR) 10-2006-0034483
Apr. 13, 2007 (KR) 10-2007-0036423

(51) Int. Cl. G01N 33/549; G01N 21/00; G01N 21/62; G01N 33/50; B29C 33/40; B29C 35/08; B29C 59/16

(52) U.S. Cl. 436/532; 422/82.05; 436/164; 436/86; 436/94; 264/219; 264/401; 264/447

(57) ABSTRACT

A bio-chip has a base plate, and a fluid resin layer positioned on the base plate and having a plurality of convexo-concave structures that are uniformly arranged in line. Side walls of the structures form reflecting films to form a Fabry-Perot interferometer structure. The bio-chip is used to detect an analyte provided to the bio-chip, so that it is possible to rapidly analyze the small amount of samples and to realize the detection sensitivity relatively higher than the conventional method.

### TECHNICAL FIELD

The present invention relates to a bio-chip, a method of manufacturing the same and a method of detecting an analyte provided to the same, and more particularly, to a bio-chip capable of detecting protein provided thereto using light interferometic, a method of manufacturing the same and a method of detecting an analyte provided to the same.

### BACKGROUND ART

In recent years, as most of human genes are revealed, it is suddenly increased interests in researches on functions of the genes constituting the human body. The genes express the protein, thereby fulfilling the functions thereof in the cells. Accordingly, the research on the genome function results in a research on the protein. Recently, a new research on proteomics revokes many interests in the inside and outside of the country. A core technology that can be necessarily used in the proteomics is a protein chip system. The protein chip system is a future chip that the core technology thereof is being researched and developed. It has a variety of application fields such as disease diagnosis, biomarker finding, research on expression and function of the protein, research on interaction of the protein, new medicine development and the like. Accordingly, it is considered that the protein chip system can be widely used in the medical science, pharmacy and life science fields.

The protein chip system is a chip in which several tens to hundreds of proteins are fixed on a substrate. Core technologies of the protein chip system are to fix the proteins on the substrate and to analyze the proteins that are fixed to the chip.

The method of analyzing the coupling of the proteins attached to the protein chip can be classified into a labeling analysis method and a non-labeling analysis method.

First, the labeling analysis method is an analysis method of measuring the intensity of fluorescence expressed by attaching a fluorescent material to the protein and quantifying the measured intensity. This method is based on a fact that the protein itself does not influence absorption and transmission properties of light. When using this method, since the fluorescent dye is selectively attached to the protein that is an analysis target, it is possible to carry out a stable analysis. However, it is troublesome to attach the fluorescent material to the protein. In addition, the culture time thereof is increased, so that the productivity is lowered.

The non-labeling analysis method is an analysis method of measuring only a mass, a refractive index or density of the protein to directly measure a concentration of the proteins attached to a protein chip system. Compared to the labeling method, the productivity is increased and a real time measurement can be made using a very small amount of sample.

As a representative method for the non-labeling method, there are an analysis method using a surface plasmon resonance (SPR) that is an optical principle and an analysis method using light interferometic. The analysis method using the SPR uses a following phenomenon: when a light wavelength is illuminated to the protein chip having the protein attached thereto, the light is not reflected in a specific wavelength but is absorbed into an analyte. In the mean time, the analysis method using the light interferometic uses a following phenomenon: when a light wavelength is illuminated to the protein chip having the protein attached thereto, there occurs an interference phenomenon on the protein chip.

When analyzing the protein analyte attached to the protein chip with the analysis method using the light interferometic, it is possible to secure sensitivity higher than with the analysis method using the SPR. In addition, the analysis method using the light interferometic is relatively simpler than the analysis method using the SPR and a light interferometic analysis apparatus is less expensive than a SPR analysis apparatus. Accordingly, it is much efficient and economic to analyze the protein analyte attached to the protein chip with the light interferometic than with the SPR.

### DISCLOSURE

### Technical Problem

Accordingly, the present invention has been made to solve the above problems. An object of the invention is to provide a bio-chip that is designed to have an optimized structure capable of using light interferometic, a method of manufacturing the same and a method of detecting an analyte provided to the same.

Another object of the invention is to provide a method of mass-producing a bio-chip with the structure capable of using light interferometic.

Still another object of the invention is to provide a bio-chip capable of achieving a high detection sensitivity and a method of manufacturing the same.

Yet still another object of the invention is to provide a method of efficiently analyzing protein attached to a bio-chip that is designed to have an optimized structure capable of using light interferometic.

### Technical Solution

In order to achieve the above object, according to an aspect of the invention, there is provided a bio-chip comprising a base plate; and a resin layer positioned on the base plate and having a plurality of convexo-concave structures that are uniformly arranged in line wherein side walls of the structures form reflecting films to form a Fabry-Perot interferometer structure.

Preferably, the base plate consists of an anti-corrosive plate and the resin layer consists of a thermosetting or UV setting resin.

In addition, a surface of the resin layer may be coated with gold (Au) or silicon carbide (SiC), silicon oxide or silicon dioxide (SiO₂).

According to the bio-chip of the invention, the side walls of the structures have an inter-wall distance (W) of 2˜50 nm and a distance (H) of 500 nm˜5 um from a bottom to an end; an inter-wall distance (W) of 10˜200 nm and a distance (H) of 1˜10 um from a bottom to an end; or an inter-wall distance (W) of 100˜2000 nm and a distance (H) of 5˜30 um from a bottom to an end.

According to another aspect of the invention, there is provided a method of manufacturing a bio-chip, which comprises preparing a stamp (nano imprint stamp) having a plurality of convexo-concave structures uniformly arranged thereto; preparing a substrate having a fluid resin on a base plate; locating and pressurizing the stamp on the fluid resin of the substrate based on a nano imprint method; setting the fluid resin of the substrate to form a resin layer having a structure corresponding to the structures of the stamp; and removing the stamp from the resin layer.

In the preparing the stamp, each side wall of the structures is preferably prepared to be parallel.

The stamp may be manufactured using a laser interferometer lithography (LIL) or e-beam lithography.

In the setting the fluid resin of the substrate, the resin layer may be formed by applying heat to the fluid resin. In this case, the stamp is preferably a heat-resistant material and the fluid resin is preferably a thermosetting resin.

In addition, in the setting the fluid resin of the substrate, the resin layer may be formed by applying ultraviolet (UV) to the fluid resin. In this case, the stamp is preferably a UV transmissive material and the fluid resin is preferably a UV setting resin.

In the preparing the substrate, the fluid resin layer may be formed on the base plate with a spin coating method.

The method may further comprise coating gold (Au) on a surface of the resin layer, alternatively may comprise coating silicon carbide (SiC), silicon oxide or silicon dioxide (SiO₂) on a surface of the resin layer, after the removing the stamp from the resin layer.

According to another aspect of the invention, there is provided a method of detecting an analyte provided to a bio-chip, which comprises preparing the bio-chip having a linker for bonding a target material; bonding the target material to the linker provided to the bio-chip; re-illuminating the bio-chip having the target material bonded thereto using light to measure a change in wavelengths caused by Fabry-Perot interferometric; and analyzing the target material bonded to the bio-chip based on the measured change in wavelengths.

Preferably, the method may further comprise illuminating the bio-chip prepared to measure Fabry-Perot interferometric resulting from patterns of the bio-chip.

The target material may be protein, nucleic acid or organic compound and the coupler for bonding the target material may include antibody, nucleic acid coupler or organic compound coupler.

A white light is preferably used as the light.

In the bonding the target material to the linker, and in the analyzing the target material, the light may be transmitted to the bio-chip through a first optic fiber and the light reflected from the bio-chip may be transmitted to a light measuring device through a second optic fiber.

In the method of detecting an analyte provided to a bio-chip, when carrying out the analysis using light in a ultraviolet region of 50˜380 nm, it is used the bio-chip having an inter-wall distance (W) of 2˜50 nm and a distance (H) of 500 nm˜5 um from a bottom to an end; when carrying out the analysis using light in a visible ray region of 380˜780 nm, it is used the bio-chip having an inter-wall distance (W) of 10˜200 nm and a distance (H) of 1˜10 um from a bottom to an end; and when carrying out the analysis using light in an infrared region of 780˜3000 nm, it is used the bio-chip having an inter-wall distance (W) of 100˜2000 nm and a distance (H) of 5˜30 um from a bottom to an end.

### ADVANTAGEOUS EFFECTS

According to the invention, followings effects are obtained.

First, it is possible to rapidly analyze the small amount of sample using the bio-chip having the light interferometer structure.

Second, it is possible to realize the detection sensitivity relatively higher than the conventional method, through the improved bio-chip structure.

Third, it is possible to mass-produce the bio-chips through the method of manufacturing the bio-chip having a predetermined pattern.

Fourth, it is possible to efficiently analyze the protein bonded to the bio-chip designed to have an optimized structure capable of using the light interferometric.

### DESCRIPTION OF DRAWINGS

The above and other objects, features and advantages of the present invention will be more apparent from the following detailed description taken in conjunction with the accompanying drawings, in which:

FIG. 1 is a perspective view of a bio-chip according to a preferred embodiment of the invention;

FIG. 2 is a flow chart illustrating a method of manufacturing a bio-chip according to a preferred embodiment of the invention;

FIG. 3 is a plan view of a stamp prepared through a method of manufacturing a bio-chip according to a preferred embodiment of the invention;

FIG. 4 is a side sectional view of a stamp prepared through a method of manufacturing a bio-chip according to a preferred embodiment of the invention;

FIG. 5 is a sectional view sequentially illustrating a method of manufacturing a bio-chip according to a preferred embodiment of the invention;

FIG. 6 is a side sectional view of a bio-chip prepared through a method of manufacturing a bio-chip according to a preferred embodiment of the invention;

FIG. 7 is a flow chart illustrating a method of detecting an analyte provided to a bio-chip according to a preferred embodiment of the invention;

FIG. 8 is a schematic diagram illustrating a method of detecting an analyte provided to a bio-chip according to a preferred embodiment of the invention;

FIG. 9 shows spectrum illustrating a change in wavelengths measured using a light measuring device when a concentration of CRP, as antigen protein, is 100 ng/ml, so as to measure the sensitivity of protein detection in a method of detecting an analyte provided to a bio-chip according to a preferred embodiment of the invention;

FIG. 10 shows spectrum illustrating a change in wavelengths measured using a light measuring device when a concentration of CRP, as antigen protein, is 10 ng/ml, so as to measure the sensitivity of protein detection in a method of detecting an analyte provided to a bio-chip according to a preferred embodiment of the invention; and

FIG. 11 shows spectrum illustrating a change in wavelengths measured through a light measuring device when a concentration of CRP, as antigen protein, is 1 ng/ml, so as to measure the sensitivity of protein detection in a method of detecting an analyte provided to a bio-chip according to a preferred embodiment of the invention.

### DESCRIPTION OF MAIN PARTS OF THE DRAWINGS

| | |
|---|---|
| 10. stamp (nano imprint stamp) | 11. convexo-concave structures |
| 20. base plate | 21. fluid resin |
| 22. resin layer | 23. gold (Au) coating layer |

### BEST MODE

Hereinafter, a preferred embodiment of the present invention will be described with reference to the accompanying drawings. The terms or words used in the specification and claims are not interpreted as typical or dictionary meanings but should be construed as meanings and concepts conforming with the technical idea of the invention, based on a principle that the inventor can properly define the concepts of the terms so as to explain the invention in the best manner. Accordingly, the embodiments described in the specification and the configurations shown in the drawings are only one preferred example of the invention, not to represent the technical idea of the invention. Therefore, it should be understood that a variety of equivalents and modifications can be made.

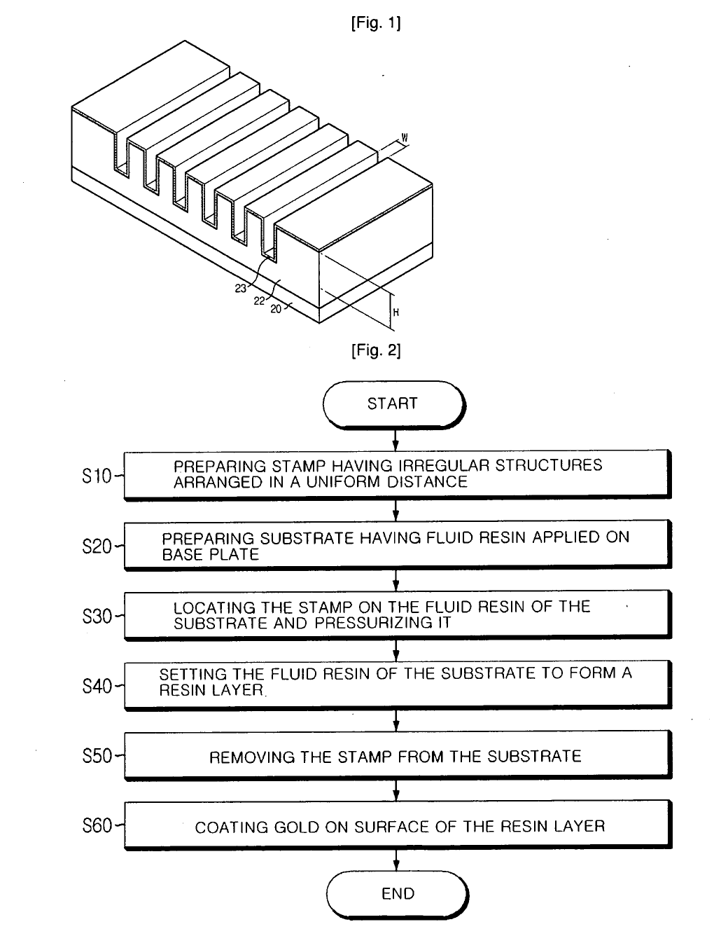

FIG. 1 is a perspective view of a bio-chip according to a preferred embodiment of the invention. Referring to FIG. 1, the bio-chip according to an embodiment of the invention comprises a base plate 20 and a resin layer 22 formed on the base plate 20.

The base plate 20 is an anti-corrosive plate and preferably consists of quartz or polymer.

The resin layer 22 is located on the base plate 20 and has a plurality of convexo-concave structures uniformly arranged. In addition, side walls of the structures of the resin layer 22 reflect light illuminated from the outside. Thereby, the side walls of the structures of the resin layer 22 form a Fabry-Perot interferometer structure.

Furthermore, when the light illuminated from the outside light is incident between the side walls of the structures of the resin layer 22, the incident light is repeatedly reflected to the side walls acting as reflecting films and then is emitted to the outside. Specifically, the light emitted to the outside causes interference by a pattern on the manufactured chip. A condition that an intensity of the light is maximized by the interference is defined with a following equation 1.

2d cos I=mλ(m=0, 1, 2 . . . ) [Math Figure 1]

where, d: inter-wall distance (W),

I: incident angle,

m: order, and

λ: wavelength of incident light.

The above equation is a condition that is satisfied when the light moves in the air. Therefore, when proteins, which have a refractive index different from that of the air, are bonded between the side walls of the convexo-concave structures of the resin layer, the light path between the side walls becomes different. As a result, since the interference patterns are different depending on a concentration difference of the proteins, it is possible to analyze the protein by detecting the change.

In order to maintain the angle of light constant, which is reflected by a surface of the resin layer 22, it is preferable to maintain the side walls of the structures flat, which are formed to the resin layer 22.

The distance and height between the side walls of the structures formed to the resin layer 22 are dependent on the wavelength region of light to be measured. It is preferable to use white light having a wide spectroscopic radiation spectrum, rather than monochromatic light, so as to observe an overall wavelength shift.

In addition, the wavelength of light can be divided into an ultraviolet region (50˜380 nm), a visible ray region (380˜780 nm) and an infrared region (780˜3000 nm). Therefore, it is preferable to use the structures of different sizes depending on the lights to be illuminated to the bio-chip and the available wavelength regions of a spectrometer measuring the lights. Specifically, when the spectrometer can analyze light in the ultraviolet region, the inter-wall distance (width: W) of the structures may be 2˜50 nm and the distance from a bottom to an end thereof (height: H) may be 500 nm˜5 um. In addition, when the spectrometer can analyze light in the visible ray region, the width (W) of the structures may be 10˜200 nm and the height (H) may be 1˜10 um. When the spectrometer can analyze a light in the infrared region, the width (W) of the structures may be 100˜2000 nm and the height (H) may be 5˜30 um.

Preferably, the surface of the resin layer 22 may be provided with a gold (Au) coating layer 23 having a high reflectivity. According to the invention, it is detected an interferometic pattern occurring while the incident light is repeatedly reflected to the side walls and then emitted to the outside. Therefore, the higher reflectivity makes the detection easier. It is known that the gold (Au) has a reflectivity of 99%. A thickness of the coating layer is preferably 20˜50 nm. The coating may be carried out in atomic layer vapor deposition or chemical vapor deposition at low temperatures. In the mean time, the gold has the high acid resistance and endurance, so that it can prevent the resin layer from being deformed. In addition, when the surface of the resin layer is coated with the gold, linkers having a thiol (—SH) group of the linkers for bonding the proteins can be easily coated to the gold surface.

In addition, the surface of the resin layer may be coated with silicon carbide (SiC), silicon oxide or silicon dioxide (SiO₂).

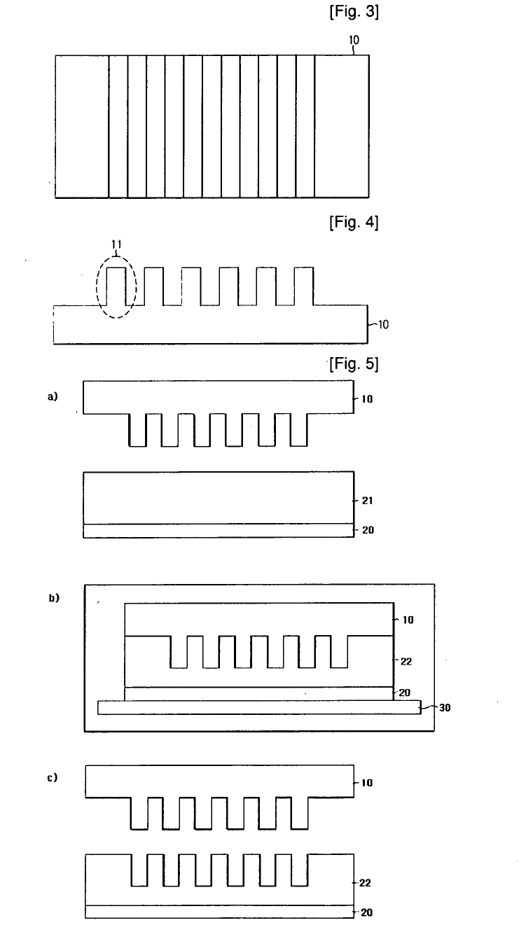

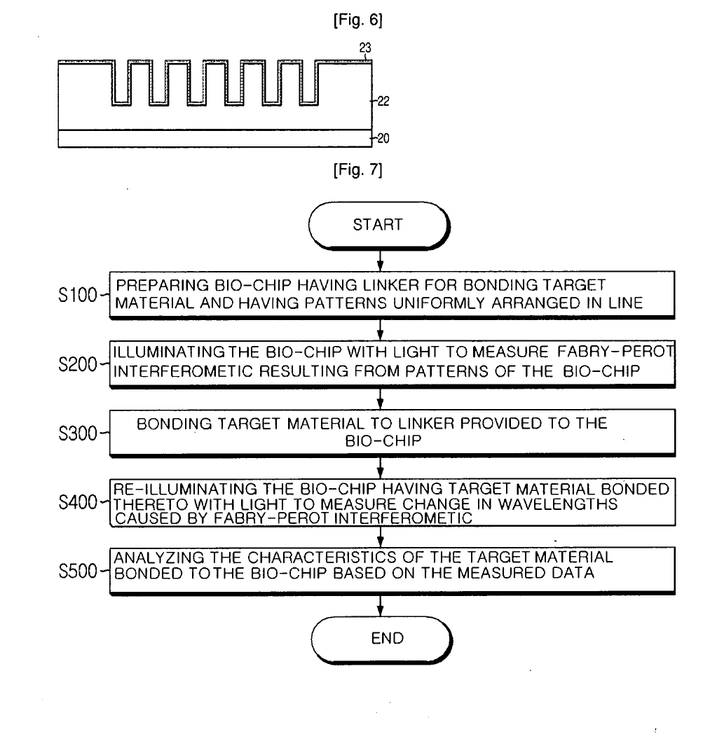

FIG. 2 is a flow chart illustrating a method of manufacturing a bio-chip according to a preferred embodiment of the invention, FIG. 3 is a plan view of a stamp prepared through a method of manufacturing a bio-chip according to a preferred embodiment of the invention, FIG. 4 is a side sectional view of a stamp prepared through a method of manufacturing a bio-chip according to a preferred embodiment of the invention, FIG. 5 is a sectional view sequentially illustrating a method of manufacturing a bio-chip according to a preferred embodiment of the invention and FIG. 6 is a side sectional view of a bio-chip prepared through a method of manufacturing a bio-chip according to a preferred embodiment of the invention.

Referring to the drawings, in the method of manufacturing a bio-chip according to an embodiment of the invention, a stamp for manufacturing a bio-chip is prepared (S10). In the S10, a plurality of periodic convexo-concave structures 11 are formed on a substrate of the stamp (nano imprint stamp) 10 using a laser interferometer lithography (LIL) or electron beam lithography (e-beam lithography). The periodic convexo-concave structures 11 are preferably formed on the substrate of the stamp (nano imprint stamp) 10 while maintaining a uniform distance.

Next, a substrate to be manufactured into a bio-chip is prepared (S20). The substrate may be manufactured by spin-coating fluid resin 21 on the base plate 20. Herein, the base plate 20 is an anti-corrosive plate and preferably consists of quartz or polymer.

Although it is exemplified that the fluid resin 21 is spin-coated on the base plate 20, the invention is not limited thereto. A method capable of forming the fluid resin 21 on the base plate 20, for example a method of applying the fluid resin 21 on the base plate 20 with an apparatus such as a dispenser can be used.

When completing the stamp (nano imprint stamp) 10 and the substrates 20, 21 of the chip, S30 is carried out. In the S30, the stamp (nano imprint stamp) 10 is located on the substrates 20, 21 of the chip, as shown in FIG. 5a. Then, as shown in FIG. 5b, heat is applied to a lower part of the base plate 20 to heat the fluid resin 21 and a predetermined pressure is applied to an upper part of the stamp (nano imprint stamp) 10. Thus, the convexo-concave structures 11, which are formed on the stamp (nano imprint stamp) 10 are imprinted on the fluid resin 21.

After the S30, the temperature of heat applied to the lower part of the base plate 20 is decreased, thereby setting the fluid resin 21 (S40). The fluid resin 21 becomes a resin layer 22 having a pattern shape corresponding to the convexo-concave structures of the stamp (nano imprint stamp) 10. At this time, the resin layer 22 should be completely set so as to prevent the pattern shape from being deformed.

In the above description, the heat is used to set the fluid resin 21 in the S40. However, the invention is not limited thereto. For example, the fluid resin 21 may be illuminated with the ultraviolet (UV) ray to set the fluid resin 21.

When the fluid resin 21 is set with the heat, it is preferable that the stamp (nano imprint stamp) 10 of heat-resistant material is prepared for the S10 and a substrate in which the fluid resin 21 consisting of thermosetting resin is coated on the base plate 20 is prepared in the S20. In the mean time, when the fluid resin 21 is set with the ultraviolet, it is preferable that the stamp (nano imprint stamp) 10 of a ultraviolet transmissive material such as quartz, glass and the like is prepared for the S10 and the UV setting resin is coated on the base plate 20 in the S20.

Next, as shown in FIG. 5c, the stamp (nano imprint stamp) 10 is removed from the resin layer 22 (S50).

After the S50, the method preferably further comprises S60 in which gold (Au) having high reflectivity, acid resistance and endurance is coated on the surface of the resin layer 22 so as to increase the reflectivity of the resin layer 22, to prevent the surface from being deformed and to enable the proteins to be fixed easily. Instead of gold (Au), silicon carbide (SiC), silicon oxide or silicon dioxide (SiO₂) may be coated on the surface of the resin layer.

Through the S30 to S60, the parts in which the convexo-concave structures 11 of the stamp exist form concave structures in the resin layer 22 and the parts in which the concave structures between the convexo-concave structures 11 exist form convex structures in the resin layer 22 (refer to FIG. 6).

Through the above processes, the resin layer 22 having the convex structures in a uniform distance are formed on the bio-chip and the structures result in a Fabry-Perot interferometic structure. In other words, the light that is illuminated from the outside and then is incident between the convex structures is repeatedly reflected to the side walls of the convex structures and then is emitted to the outside. Therefore, when protein samples are bonded between the convex structures, it is possible to analyze the protein through the analysis of the wavelength of the light illuminated from the outside and the wavelength of the light emitted to the outside.

In order to maintain the angle of light constant, which is reflected from the surface of the resin layer 22, the side walls of the structures formed in the resin layer 22 should be maintained to be parallel. To this end, it is preferable that the side walls of the convexo-concave structures 11 of the stamp (nano imprint stamp) 10 are formed to be parallel in the S10.

In addition, as described above, the sizes of the structures of the resin layer 22 are determined depending on the wavelength regions (for example, ultraviolet region, visible ray region or infrared region) that can be analyzed by a spectrometer. The sizes of the convexo-concave structures 11 for forming the above structures in the resin layer 22 should be also prepared in accordance with the determined size.

FIG. 7 is a flow chart illustrating a method of detecting an analyte provided to a bio-chip according to a preferred embodiment of the invention and FIG. 8 is a schematic diagram illustrating a method of detecting an analyte provided to a bio-chip according to a preferred embodiment of the invention. In the followings, a method of detecting an analyte provided to a bio-chip will be described with reference to FIGS. 7 and 8.

First, in order to bond a target material to a bio-chip having patterns uniformly arranged in line, it is prepared a bio-chip having a linker (S100).

Next, the bio-chip prepared in the S100 is illuminated with the light to measure a Fabry-Perot interferometic resulting from the patterns of the bio-chip (S200).

At this time, the light is illuminated and the reflected light is measured using a probe having first and second optic fibers. Specifically, when the light is illuminated to the first optic fiber of the probe, instead of directly illuminating the light to the bio-chip, the first optic fiber acting as light transmitting medium transmits to the bio-chip the light in a specific wavelength transmitted from the light. In addition, the light reflected from the bio-chip is also transmitted to the second optic fiber which in turn transmits the light to the light measuring device.

In the mean time, it is preferable to use white light having a wide spectroscopic radiation spectrum, rather than monochromatic light, so as to observe an overall wavelength shift.

Preferably, the first and second optic fibers are provided to a single probe. In addition, the probe may be provided with plural first and second optic fibers.

When completing the S200, the target material is bonded to the linker provided to the bio-chip (S300).

Preferably, the target material is protein and the coupler for bonding the material to the bio-chip is antibody.

Although the protein is exemplified as the target material and the antibody is exemplified as the coupler for bonding the material to the bio-chip, the invention is not limited thereto. For example, the target material may be an organic compound such as nucleic acid. In this case, the coupler may be an organic compound such as nucleic acid coupler.

When the target material is bonded to the bio-chip through the S300, the bio-chip having the target material bonded thereto is re-illuminated using the light and the probe used in the S200 and a wavelength shift resulting from the Fabry-Perot interferometic is measured with the light measuring device (S400).

Finally, based on the data measured in the S200 and S400, the characteristics of the target material bonded to the bio-chip (S500) is analyzed.

Although it is described that the S300 is carried out between the S100 and S300, the invention is not limited thereto. In other words, the S300 may be carried out anytime before the S500.

The data analyzed through the S100 to S500 can be used for disease diagnosis, biomarker finding, research on expression and function of the proteins, research on interaction of the protein, new medicine development and the like.

### MODE FOR INVENTION

In the following examples, the proteins were actually attached to the bio-chip and detected, so that the sensitivity of the detection was checked. First, the linker was vapor-deposited on a resin layer surface coated with gold (Au). Prolinker™ available from Proteogen Company was used as the linker. Then, the anti-CRP antibody was coupled to the linker. The coupling was made as the linker recognized —NH₃⁺ of the protein. After the antibody was coupled, the blocking was carried out so as to prevent the proteins from being non-specifically bonded to other than the antibody. Then, the antigen CRP, as target protein, was bonded.

A HR-4000 (Ocean Optics) spectrometer was used as the light measuring device. A halogen lamp was used as the light source. The wavelength of the light used for the analysis was within a range of 350˜850 nm. The side walls of the structures on the chip had the inter-wall distance (W) of 40˜60 nm and the distance (H) of 1.5 um from bottom to end.

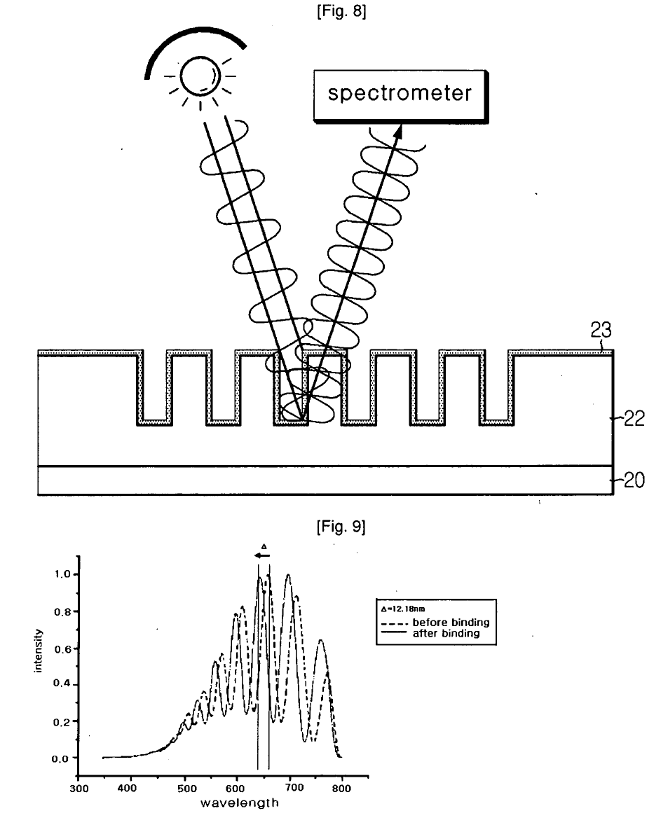

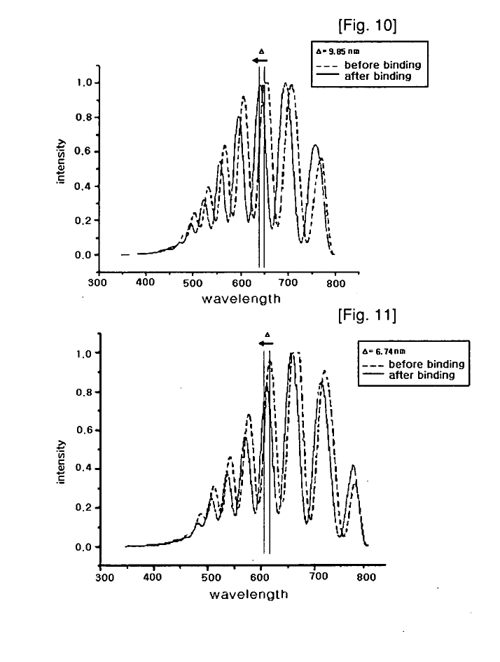

FIGS. 9 to 11 show spectrums illustrating the measurement results of protein detection sensitivity while changing a concentration of CRP that is antigen protein into 100 ng/ml (FIG. 9), 10 ng/ml (FIG. 10) and 1 ng/ml (FIG. 11). Each spectrum shows that the spectrum (solid line) after the antigen is bonded is much shifted (Δ) to the left, as compared to the spectrum (dotted line) before the antigen is bonded in antibody binding. This is an effect caused by the interferometic and shows that the detection sensitivity is excellent, i.e., the protein can be detected up to 1 ng/ml.

While the invention has been shown and described with reference to certain preferred embodiments thereof, it will be understood by those skilled in the art that various changes in form and details may be made thereto without departing from the spirit and scope of the invention as defined by the appended claims.

### INDUSTRIAL APPLICABILITY

As described above, according to the invention, it is possible to rapidly analyze the small amount of sample using the bio-chip having the light interferometer structure, to realize the detection sensitivity relatively higher than the conventional method, through the improved bio-chip structure to mass-produce the bio-chips through the method of manufacturing the bio-chip having a predetermined pattern and to efficiently analyze the protein bonded to the bio-chip designed to have an optimized structure capable of using the light interferometric.

1. A bio-chip comprising:
a base plate; and
a resin layer positioned on the base plate and having a plurality of convexo-concave structures that are uniformly arranged in line,
wherein side walls of the structures form reflecting films to form a Fabry-Perot interferometer structure.

2. The bio-chip according to claim 1, wherein the base plate is an anti-corrosive plate.

3. The bio-chip according to claim 1, wherein the resin layer consists of a polymeric resin.

4. The bio-chip according to claim 3, wherein the polymeric resin is a thermosetting resin.

5. The bio-chip according to claim 3, wherein the polymeric resin is an UV setting resin.

6. The bio-chip according to claim 1, wherein a surface of the resin layer is coated with gold (Au) layer.

7. The bio-chip according to claim 1, wherein a surface of the resin layer is coated with a silicon carbide (SiC), silicon oxide or silicon dioxide (SiO₂).

8. The bio-chip according to claim 1, the side walls of the structures have an inter-wall distance (W) of 2˜50 nm and a distance (H) of 500 nm˜50 um from a bottom to an end.

9. The bio-chip according to claim 1, the side walls of the structures have an inter-wall distance (W) of 10˜200 nm and a distance (H) of 1˜10 um from a bottom to an end.

10. The bio-chip according to claim 1, the side walls of the structures have an inter-wall distance (W) of 100˜2000 nm and a distance (H) of 5˜30 um from a bottom to an end.

11. A method of manufacturing a bio-chip, comprising:
preparing a stamp having a plurality of convexo-concave structures uniformly arranged thereon;
preparing a substrate having a fluid resin on a base plate;
locating and pressurizing the stamp on the fluid resin of the substrate based on a nano imprint method;
setting the fluid resin of the substrate to form a resin layer having a structure corresponding to the structures of the stamp; and
removing the stamp from the resin layer.

12. The method according to claim 11, wherein in the preparing the stamp, each side wall of the structures is prepared to be parallel.

13. The method according to claim 11, wherein in the setting the fluid resin of the substrate, the resin layer is formed by applying heat to the fluid resin.

14. The method according to claim 11, wherein in the setting the fluid resin of the substrate, the resin layer is formed by applying ultraviolet to the fluid resin.

15. The method according to claim 11, further comprising coating gold (Au) on a surface of the resin layer after the removing the stamp from the resin layer.

16. The method according to claim 11, further comprising coating silicon carbide (SiC), silicon oxide or silicon dioxide (SiO₂) on a surface of the resin layer, after the removing the stamp from the resin layer.

17. The method according to claim 11, wherein the stamp is manufactured with a laser interferometer lithography (LIL).

18. The method according to claim 11, wherein the stamp is manufactured with an electron beam lithography.

19. The method according to claim 11, wherein the base plate is an anti-corrosive plate.

20. The method according to claim 11, wherein the fluid resin is a polymeric resin.

21. The method according to claim 13, wherein the resin is a thermosetting resin.

22. The method according to claim 14, wherein the resin is an UV setting resin.

23. The method according to claim 11, wherein in the preparing the substrate, the fluid resin layer is formed on the base plate with a spin coating method.

24. A method of detecting an analyte provided to a bio-chip, comprising:
preparing the bio-chip according to claim 1, having a linker for bonding a target material;
bonding the target material to the linker provided to the bio-chip;
re-illuminating the bio-chip having the target material bonded thereto using light to measure a change in wavelengths caused by Fabry-Perot interferometric; and
analyzing the target material bonded to the bio-chip based on the measured change in wavelengths.

25. The method according to claim 24, further comprising
illuminating light to the prepared bio-chip to measure Fabry-Perot interferometric resulting from patterns of the bio-chip.

26. The method according to claim 24, wherein the target material is protein.

27. The method according to claim 26, wherein in the bonding protein to the linker, a coupler for bonding protein is further used and the coupler is an antibody.

28. The method according to claim 24, wherein the target material is nucleic acid.

29. The method according to claim 28, wherein in the bonding nucleic acid to the linker, a coupler for bonding nucleic acid is further used and the coupler is an nucleic acid coupler.

30. The method according to claim 24, wherein the target material is an organic compound.

31. The method according to claim 30, wherein in the bonding organic compound to the linker, a coupler for bonding organic compound is further used and the coupler is an organic compound coupler.

32. The method according to claim 24, wherein the light is white light.

33. The method according to claim 24, wherein in the analyzing the target material, the light is transmitted to the bio-chip through a first optic fiber and the light reflected from the bio-chip is transmitted to a light measuring device through a second optic fiber.

34. The method according to claim 24, wherein the bio-chip according to claim 8 is used when carrying out the analyzing using light in a ultraviolet region of 50˜380 nm.

35. The method according to claim 24, wherein the bio-chip according to claim 9 is used when carrying out the analyzing using light in a visible ray region of 380˜780 nm.

36. The method according to claim 24, wherein the bio-chip according to claim 10 is used when carrying out the analyzing using light in an infrared region of 780˜3000 nm.

37. The method according to claim 20, wherein the resin is a thermosetting resin.

38. The method according to claim 20, wherein the resin is an UV setting resin.

39. The method according to claim 25, wherein the bio-chip according to claim 8 is used when carrying out the analyzing using light in a ultraviolet region of 50˜380 nm.

40. The method according to claim 25, wherein the bio-chip according to claim 9 is used when carrying out the analyzing using light in a visible ray region of 380˜780 nm.

41. The method according to claim 25, wherein the bio-chip according to claim 10 is used when carrying out the analyzing using light in an infrared region of 780˜3000 nm.
